(19) United States
(12) Patent Application Publication
(10) Pub. No.: US 2012/0316413 A1
(43) Pub. Date: Dec. 13, 2012

(54) MEASUREMENT APPARATUS

(75) Inventors: Shuhai Liu, Beijing (CN); Feng Xu, Beijing (CN); Weihu Wang, Beijing (CN); Peng Wu, Beijing (CN); Yanqing Zhang, Beijing (CN)

(73) Assignee: BEIJING CHOICE ELECTRONIC TECHNOLOGY CO., LTD., Beijing (CN)

(21) Appl. No.: 13/514,471

(22) PCT Filed: Jan. 18, 2011
(86) PCT No.: PCT/CN2011/070366

§ 371 Date: Jun. 7, 2012

(51) Int. Cl. A61B 5/00; A61B 5/0402; A61B 5/02; A61B 5/01; A61B 5/0408; A61B 5/145

(52) U.S. Cl. 600/364; 600/300; 600/393; 600/508; 600/549; 600/509

(57) ABSTRACT

Disclosed is a measurement apparatus that includes a main body, a signal processing module disposed inside the main body, a first data communication interface coupled with the signal processing module, and a signal collection module coupled with the signal processing module; the signal collection module functions to measure the physiological parameter signal and output the physiological parameter signal to the signal processing module; the signal processing module functions to process the physiological parameter signal to generate the physiological parameter data, and output the physiological parameter data to the terminal device via the first data communication interface, such that the terminal device can display the physiological parameter data. The user can directly conduct the physiological parameter measurement using the measurement apparatus according to the present invention without any specialized measurement equipment.

### TECHNICAL FIELD

The present invention relates to the filed of medical detecting technology, and more particularly relates to a measurement apparatus.

### BACKGROUND ART

Various diseases that may threaten human healthy and life are increasing in modern society. Currently, most of the diseases are treated by preventing and observing. The medical personnel measure various physiological parameters of the patients using various measurement apparatus so as to obtain various data information related to the patients. The measurement apparatus may be the electrocardiograph instrument, the oximeter or thermometer or the temperature detector etc. Take the heart disease as an example, the medical personnel can measure the electrocardiogram of the patient using the electrocardiograph instrument, and can rapidly acquire the relevant information of the heart of the patient by observing the form of the electrocardiograph.

For facilitating detection and record of the physical states at any time for the patient, a plurality of hand-held measurement apparatus for measuring various physiological parameters specifically have been developed by the persons skilled in the art. In the following, the hand-held electrocardiograph instrument will be described as an example. FIG. 1 is a schematic view of a structure of the hand-held electrocardiograph instrument in the prior art. As shown in FIG. 1, the hand-held electrocardiograph instrument has a display screen 1 in the front side for displaying the electrocardiograph, a control key set 2 is disposed at the side of the display screen 1; contact electrodes 3 and 4 are disposed on the left and right sides of the plane that the display screen 1 is located, respectively; and an electrode interface 5 is disposed at a side below the plane, that the display screen 1 is located, for connecting an external electrode. The hand-held electrocardiograph instrument has many measuring manners, including: 1, hands measuring, wherein the two hands contact the contact electrodes 3 and 4 at the opposite sides of the electrocardiograph instrument for measuring the electrocardiograph signals of the hands; 2, hands and ankles measuring, wherein the right hand and the left ankle (or the left hand and the right ankle) respectively contact the contact electrodes 3 and 4 at the opposite sides of the electrocardiograph instrument for measuring the electrocardiograph signals of the ankles and the hands; 3, hands and chest measuring, wherein the right hand contacts the contact electrode 3 at the right side of the electrocardiograph instrument, and the contact electrode 4 at the left side of the electrocardiograph instrument is disposed below the left chest, so as to measure the electrocardiograph signals of the hands and the chest; 4, external electrode measuring, wherein three external surface mounted electrodes are firstly connected to the electrode interface 5, and then the external electrodes are respectively adhered below the left/right clavicles and at the left lower abdomen for measuring the electrocardiograph signals of these three positions.

It can be seen from the prior art hand-held electrocardiograph instrument that the specialized hand-held measurement apparatus can fulfill the requirements of measuring vaious physiological parameters of the patient himself in some extent. However, the following drawbacks exist in the prior art hand-held measurement apparatus inevitably: such a hand-held measurement apparatuses is a specialized equipment with high price, not every user can bear such price; and the hand-held measurement apparatus has large body which is not convenient for the user to carry. Therefore, user cannot carry out the measurement of the physiological parameters at any time in the prior art.

### SUMMARY

The present invention provides a measurement apparatus to solve the problem that users have difficulty in measuring the physiological parameters at any time and at any place.

To achieve this object, the present invention provides a measurement apparatus comprising: a main body, a signal processing module disposed inside the main body, a first data communication interface coupled with the signal processing module, and a signal collection module coupled with the signal processing module;

the first data communication interface being coupled with a terminal device;

the signal collection module functions to measure the physiological parameter signal and output the physiological parameter signal to the signal processing module;

the signal processing module functions to process the physiological parameter signal to generate the physiological parameter data, and output the physiological parameter data to the terminal device via the first data communication interface, such that the terminal device can display the physiological parameter data.

Furthermore, the main body includes a bottom plate and sides at edges of the bottom plate.

Furthermore, the sides have an internal hollow structure, and the signal processing module is disposed inside the sides.

Furthermore, a projection portion is provided at the outside of the bottom plate, and the signal processing module is disposed inside the projection portion.

Furthermore, the sides and the bottom plate form a cavity, and the main body is nested outside of the terminal device through the cavity.

Furthermore, the sides surround at the edges of the bottom plate.

Furthermore, the measurement apparatus further includes a first cover member with an annular shape, and the first cover member is clasped on the sides.

Furthermore, chutes are provided on two opposite edges of the bottom plate, and flanges matching with the chutes are provided at the two opposite edges of the bottom plate, the sides slide along the chutes through the flanges.

Furthermore, the sides are provided at one edge and a part of an edge adjacent to the one edge of the bottom plate.

Furthermore, the sides are provided at the two opposite edges of the bottom plate.

Furthermore, one side of the bottom plate has an open.

Furthermore, the main body has a second cover member, and the second cover member is clasped on the open.

Furthermore, the signal collection module includes at least two contact electrodes, and the contact electrodes are disposed on the second cover member;

A first conductive member is disposed at the edge contacting the open on the second cover member, and a second conductive member is disposed on the open, the second conductive member is connected to the signal processing module through wires, the first conductive member is in contact with the second conductive member so as to achieve the electrical connection between the contact electrode on the second cover member and the signal processing module.

Furthermore, the main body is a shell, the signal processing module is disposed inside the shell.

Furthermore, the measurement apparatus further includes a power supply module which is disposed inside the main body and connected to the signal processing module;

the power supply module functions to supply power to the signal processing module.

Furthermore, the first data communication interface is disposed inside the main body, and the first data communication interface is a wireless interface.

Furthermore, the signal collection module includes at least two contact electrodes, and the physiological parameter signal includes the electrocardiograph signal, the physiological parameter data includes the electrocardiograph data, and the signal processing module includes an electrocardiograph processing sub-module;

the contact electrodes function to measure the electrocardiograph signal, and output the electrocardiograph signal to the electrocardiograph processing sub-module;

the electrocardiograph processing sub-module functions to process the electrocardiograph signal to generate the electrocardiograph data, and output the electrocardiograph data to the terminal device through the first data communication interface, such that the terminal device can display the electrocardiograph data.

Furthermore, three contact electrodes are disposed at the outside of the bottom plate, and the contact electrodes are arranged in an isosceles triangle shape.

Furthermore, the contact electrodes are disposed at the outside of the sides.

Furthermore, the contact electrodes are connected to the signal processing module through wires, and the sides have an internal hollow structure, and the wires are disposed inside the sides.

Furthermore, the signal collection module includes an external detection device, and the measurement apparatus further includes: a second data communication interface which is disposed on the main body and is connected to the signal processing module, the second data communication interface functions to connect the external detection device;

the external detection device functions to measure the physiological parameter signal and output the physiological parameter signal to the signal processing module through the second data communication interface.

Furthermore, the external detection device is a blood oxygen measurement module, the physiological parameter signal includes the blood oxygen signal, the physiological parameter data includes the blood oxygen data, and the signal processing module includes a blood oxygen processing sub-module;

the blood oxygen measurement module functions to measure the blood oxygen signal and output the blood oxygen signal to the blood oxygen processing sub-module;

the blood oxygen processing sub-module functions to process the blood oxygen signal to generate the blood oxygen data, and output the blood oxygen data to the terminal device through the first data communication interface, such that the terminal device can display the blood oxygen data.

Furthermore, the external detection device is a fetal heart measurement module, and the physiological parameter signal includes the fetal heart signal, the physiological parameter data includes the fetal heart data, and the signal processing module includes a fetal heart processing sub-module;

the fetal heart measurement module functions to measure the fetal heart signal, and output the fetal heart signal to the fetal heart processing sub-module;

the fetal heart processing sub-module functions to process the fetal heart signal to generate the fetal heart data, and output the fetal heart data to the terminal device through the first data communication interface, such that the terminal device can display the fetal heart data.

Furthermore, the external detection device is a temperature measurement module, the physiological parameter signal includes the temperature signal, the physiological parameter data includes the temperature data, and the signal processing module includes a temperature processing sub-module;

the temperature measurement module functions to measure the temperature signal and output the temperature signal to the temperature processing sub-module;

the temperature processing sub-module functions to process the temperature signal to generate the temperature data, and output the temperature data to the terminal device through the first data communication interface, such that the terminal device can display the temperature data.

Furthermore, the external detection device is an inductive electrode, the physiological parameter signal includes the electrocardiograph signal, the physiological parameter data includes the electrocardiograph data, and the signal processing module includes the electrocardiograph processing sub-module;

the inductive electrode functions to measure the electrocardiograph signal, and

output the electrocardiograph signal to the electrocardiograph processing sub-module;

the electrocardiograph processing sub-module functions to process the electrocardiograph signal to generate the electrocardiograph data, and output the electrocardiograph data to the terminal device through the first data communication interface, such that the terminal device can display the electrocardiograph data.

Furthermore, the second data communication interface is disposed inside the main body, and the second data communication interface is a wireless interface.

The present invention can provide the following advantageous effects:

The present invention provides a measurement apparatus comprising the main body, the signal processing module disposed inside the main body, the first data communication interface connected to the signal processing module and the signal collection module connected to the signal processing module, the signal collection module outputs the measured physiological parameter signal to the signal processing module, the signal processing module process the physiological parameter signal to generate the physiological parameter data and output the physiological parameter data to the terminal device through the first data communication interface, such that the terminal device can display the physiological parameter data. The user can directly conduct the physiological parameter measurement using the measurement apparatus according to the present invention without any specialized measurement equipment. The measurement apparatus according to the present invention together with various hand-held terminal devices carried at any time can implement the physiological parameter measurement at any time and at any place.

### BRIEF DESCRIPTION OF THE DRAWINGS

FIG. 1 is a schematic view showing the structure of the prior art hand-held electrocardiograph instrument;

FIG. 2 is the front view of the structure of a measurement apparatus according to a first embodiment of the present invention;

FIG. 3 is the back-side view of the structure of the measurement apparatus in FIG. 2;

FIG. 4 is a schematic view showing an application of the measurement apparatus in FIG. 2;

FIG. 6 is the front view of the structure of a measurement apparatus according to a second embodiment of the present invention;

FIG. 7 is the back-side view of the structure of the measurement apparatus in FIG. 6;

FIG. 8 is a schematic view showing an application of the measurement apparatus in FIG. 6;

FIG. 9 is the front view of the structure of a measurement apparatus according to a third embodiment of the present invention;

FIG. 10 is the back-side view of the structure of the measurement apparatus in

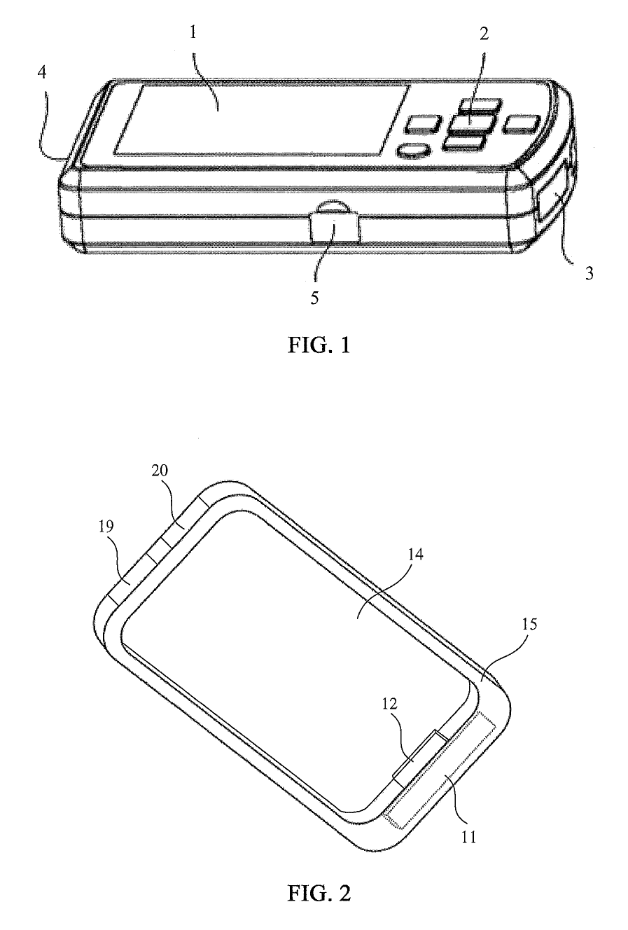

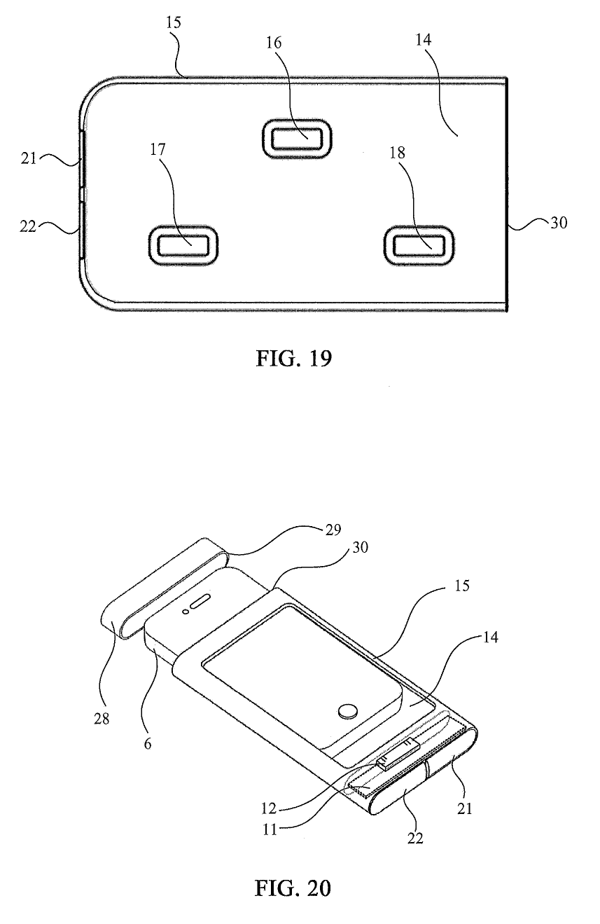

FIG. 9;

FIG. 11 is a schematic view showing an application of the measurement apparatus in FIG. 9;

FIG. 12 is the front view of the structure of a measurement apparatus according to a fourth embodiment of the present invention;

FIG. 13 is the back-side view of the structure of the measurement apparatus in FIG. 12;

FIG. 14 is a schematic view showing an application of the measurement apparatus in FIG. 12;

FIG. 15 is the front view of the structure of a measurement apparatus according to a fifth embodiment of the present invention;

FIG. 16 is the back-side view of the structure of the measurement apparatus in FIG. 15;

FIG. 17 is a schematic view showing an application of the measurement apparatus in FIG. 15;

FIG. 18 is the front view of the structure of a measurement apparatus according to a sixth embodiment of the present invention;

FIG. 19 is the back-side view of the structure of the measurement apparatus in FIG. 18;

FIG. 20 is a schematic view showing an application of the measurement apparatus in FIG. 18;

FIG. 21 is the front view of the structure of a measurement apparatus according to a seventh embodiment of the present invention;

FIG. 22 is a schematic view showing an application of the measurement apparatus in FIG. 21.

### DETAILED DESCRIPTION OF THE PREFERRED EMBODIMENTS

To provide a better understanding of the technical solution of the present invention for the persons skilled in the art, the measurement apparatus of the present invention will be described in detail with reference to the accompanying drawings.

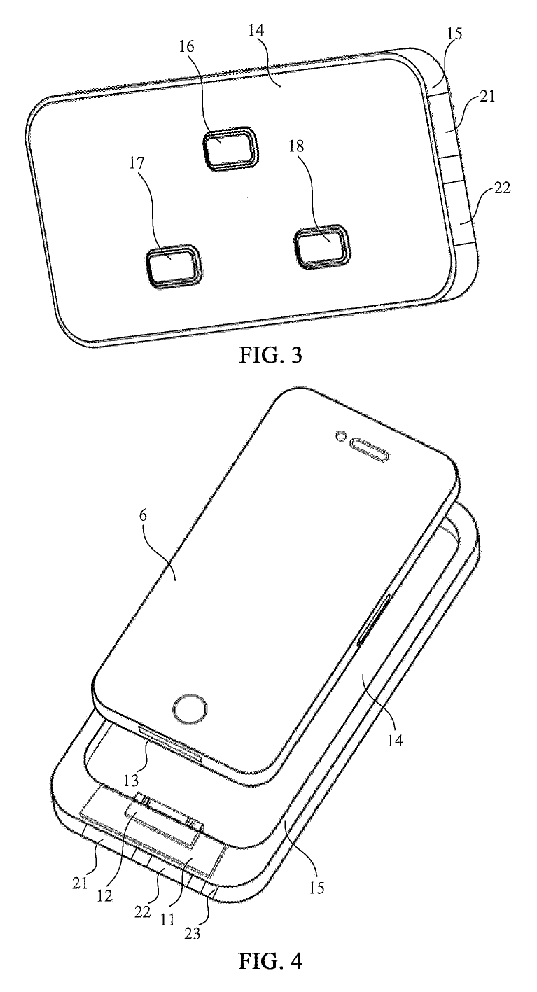

FIG. 2 is the front view of the structure of a measurement apparatus according to a first embodiment of the present invention, FIG. 3 is the back-side view of the structure of the measurement apparatus in FIG. 2, and FIG. 4 is a schematic view showing an application of the measurement apparatus in FIG. 2. As shown in FIGS. 2-4, the measurement apparatus includes: a main body, a signal processing module 11 disposed inside the main body, a first data communication interface 12 coupled with the signal processing module 11, and a signal collection module coupled with the signal processing module 11. The first data communication interface 12 is coupled with a terminal device 6; the signal collection module functions to measure the physiological parameter signal and output the physiological parameter signal to the signal processing module 11; the signal processing module 11 functions to process the physiological parameter signal to generate the physiological parameter data, and output the physiological parameter data to the terminal device via the first data communication interface 12, such that the terminal device 6 can display the physiological parameter data.

In the present invention, the terminal device 6 can be a portable intelligent device with display functions, which intelligent device can load software. For example, the terminal device 6 can be a mobile phone, a computer, MP4 or MP3. The present embodiment takes the mobile phone as an example to describe the technical solution. Specifically, the terminal device 6 loads a software capable of displaying the physiological parameter data. When the terminal device 6 receives the physiological parameter data, the software can display the physiological parameter data as a table on the display screen, such that the user can access the physiological parameter data through the terminal device 6.

In the present invention, the first data communication interface 12 can be an interface which matches the terminal communication interface 13 of the terminal device 6. The first data communication interface 12 is disposed partly outside the main body so as to be connected with the terminal communication interface 13. The first data communication interface 12 can be a standard interface, e.g. a USB interface, or a specialized interface for a certain terminal device.

The main body includes a bottom plate 14 and the sides 15 provided at the edges of the bottom plate 14. The shape of the bottom plate 14 and the sides 15 can be designed according to that of the terminal device 6. Preferably, the bottom plate 14 is flat. In the present embodiment, the shape of the bottom plate 14 is a rectangle with chamfers. Preferably, the sides 15 have an internal hollow structure, and the signal processing module 11 can be disposed inside the sides 15. The sides 15 and the bottom plate 14 form a cavity, the main body is set outside the terminal device 6 by the cavity which accommodates the terminal device 6. The display screen of the terminal device 6 has its back on the bottom plate 14. The sides 15 surround at the edges of the bottom plate 14. In the present embodiment, the sides 15 have a structure of surrounding the edges of the bottom plate 14 continuously.

Further, in the present embodiment, the signal collection module includes two contact electrodes, and the physiological parameter signals include the electrocardiograph signals, and the physiological parameter data includes the electrocardiograph data. FIG. 5 is a schematic view of the structure of the signal processing module of the present invention. As shown in FIG. 5, the signal processing module 11 includes an electrocardiograph processing sub-module 111. The contact electrodes function to measure the electrocardiograph signal, and output the electrocardiograph signal to the electrocardiograph processing sub-module 111; the electrocardiograph processing sub-module 111 functions to process the electrocardiograph signal, generate the electrocardiograph data, and output the electrocardiograph data to the terminal device 6 through the first data communication interface 12, such that the terminal device 6 can display the electrocardiograph data. The contact electrodes are disposed at the outside of the bottom plate 14 or at the outside of the sides 15. In the present invention, “inside” refers to the side facing the cavity, and accordingly “outside” refers to the side that back on the cavity. The number of the contact electrodes can be at least two. For example: in the present embodiment, there are three contact electrodes disposed at the outside of the bottom plate 14, i.e. contact electrodes 16, 17 and 18, which are arranged in an isosceles triangle shape. Specifically, the bottom plate 14 can also has an internal hollow structure, and the internal hollow structure of the bottom plate 14 and that of the sides 15 communicate with each other, such that the contact electrodes disposed at the outside of the bottom plate 14 can be connected to the signal processing module 11 through conducting wires provided in the bottom plate 14 and the sides 15 (not shown). Preferably, the conducting wires of the present invention have insulated shell. Contact electrodes can be provided at the outside of the sides 15. In the present embodiment, there are four contact electrodes disposed at the outside of the sides 15, i.e. the contact electrodes 19, 20, 21 and 22, wherein the contact electrodes disposed at the sides 15 can be connected to the signal processing module 11 through conducting wires provided in the sides 15 (not shown). Furthermore, the contact electrodes disposed at the outside of the sides 15 can also be disposed at the outside of the sides 15 on the other edges of the bottom plate 14, and the description thereof is omitted herein. The contact electrodes are mutually insulated.

In the present invention, the signal processing module 11 can be directly powered by the terminal device 6 which is connected to the measurement apparatus. In particular, when the first data communication interface 12 is connected with the terminal communication interface 13, the terminal device 6 can power the signal processing module 11 through the terminal communication interface 13 and the first data communication interface 12. Alternatively, the measurement apparatus can further include a power supply module provided inside the main body and connected with the signal processing module 11, and the signal processing module 11 is powered by the power supply module. For example, the power supply module can be disposed inside the sides 15 to be in proximity to the signal processing module 11, and is connected to the signal processing module 11 through conducting wires (not shown). Preferably, the power supply module can be a button cell.

Furthermore, in the present embodiment, the signal collection module includes an external detection device, and the measurement apparatus further includes: a second data communication interface 23 which is disposed on the main body and is connected to the signal processing module 11, the second data communication interface 23 functions to connect the external detection device. The external detection device functions to measure the physiological parameter signals and to output the physiological parameter signals to the signal processing module 11 through the second data communication interface 23. The external detection device can be plugged into the second data communication interface 23 directly, at this time the communication interface of the external detection device needs to match the second data communication interface 23, the external detection device is not shown in the figures. The second data communication interface 23 can be a standard interface, e.g. a USB interface, or a specialized interface for a certain external detection device.

Optionally, the external detection device is a blood oxygen measurement module, the physiological parameter signal includes the blood oxygen signal and the physiological parameter data includes the blood oxygen data. As shown in FIG. 5, the signal processing module 11 includes a blood oxygen processing sub-module 112. The blood oxygen measurement module functions to measure the blood oxygen signal and output the blood oxygen signal to the blood oxygen processing sub-module 112; the blood oxygen processing sub-module 112 functions to process the blood oxygen signal, generate the blood oxygen data, and output the blood oxygen data to the terminal device 6 through the first data communication interface 12, such that the terminal device 6 can display the blood oxygen data. For example, the blood oxygen measurement module can be a blood oxygen probe.

Optionally, the external detection device is a fetal heart measurement module, and the physiological parameter signal includes the fetal heart signal, and the physiological parameter data includes the fetal heart data. As shown in FIG. 5, the signal processing module 11 includes a fetal heart processing sub-module 113. The fetal heart measurement module functions to measure the fetal heart signal, and output the fetal heart signal to the fetal heart processing sub-module 113; the fetal heart processing sub-module 113 functions to process the fetal heart signal, generate the fetal heart data, and output the fetal heart data to the terminal device 6 through the first data communication interface 12, such that the terminal device 6 can display the fetal heart data. For example, the fetal heart measurement module can be a fetal heart Doppler probe.

Optionally, the external detection device is a temperature measurement module, the physiological parameter signal includes the temperature signal, and the physiological parameter data includes the temperature data. As shown in FIG. 5, the signal processing module 11 includes a temperature processing sub-module 114. The temperature measurement module functions to measure the temperature signal and output the temperature signal to the temperature processing sub-module 114; the temperature processing sub-module 114 functions to process the temperature signal, generate the temperature data, and output the temperature data to the terminal device 6 through the first data communication interface 12, such that the terminal device 6 can display the temperature data. For example, the temperature measurement module can be a temperature detector such as an infrared non-contact temperature measurement device or a metal heat-conducting temperature measurement device.

Optionally, the external detection device is an inductive electrode, the inductive electrode functions to measure the electrocardiograph signal, and output the electrocardiograph signal to the electrocardiograph processing sub-module; the electrocardiograph processing sub-module 111 functions to process the electrocardiograph signal, generate the electrocardiograph data, and output the electrocardiograph data to the terminal device 6 through the first data communication interface 12, such that the terminal device 6 can display the electrocardiograph data. For example, the inductive electrode includes a lead wire, the electrode plate is disposed at one end of the lead wire, and the other end of the lead wire is connected to the second data communication interface 23.

Furthermore, the signal processing module 11 can be provided at other positions in the main body. For example, the bottom plate 14 can have an internal hollow structure, and the signal processing module 11 can be disposed inside the bottom plate 14; or the signal processing module 11 can be located inside the sides 15 on the other edges of the bottom plate 14 shown in the figures, which is not depicted in the figures; or the signal processing module 11 can provide a protrusion portion at the outside of the bottom plate 14 and the signal processing module 11 is disposed inside the protrusion portion, which is not depicted in the figures. The height of the protrusion portion is selected as not affecting the measurement performed by the contact electrodes. Preferably, the height of the protrusion portion is smaller than those of the contact electrodes on the bottom plate.

In practice, the terminal device 6 is disposed in the cavity of the measurement apparatus, such that the measurement apparatus is sheathed outside the terminal device 6, and the terminal communication interface 13 is connected to the first data communication interface 12. For example, the terminal communication interface 13 can be plugged into the first data communication interface 12 directly, and then the user can measure the electrocardiograph by the measurement apparatus.

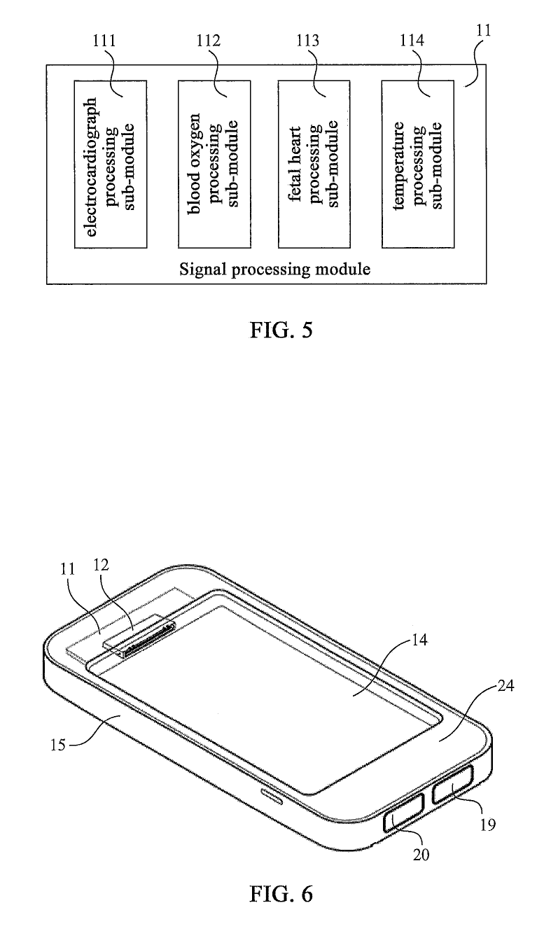

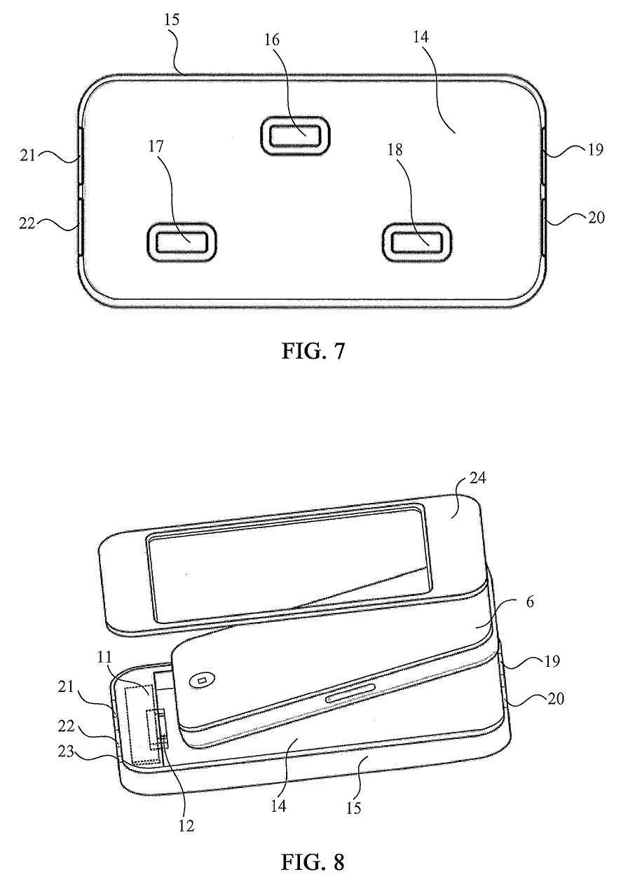

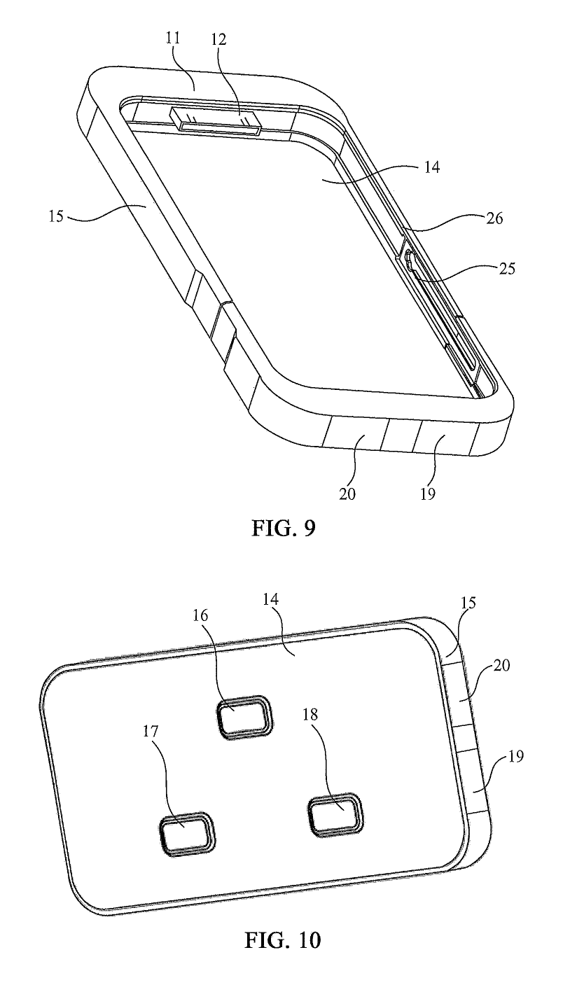

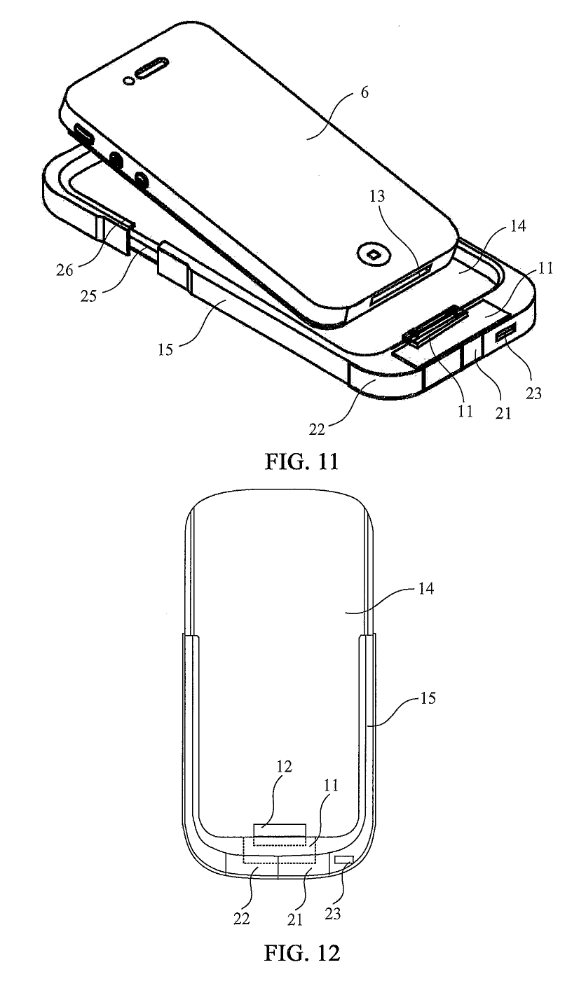

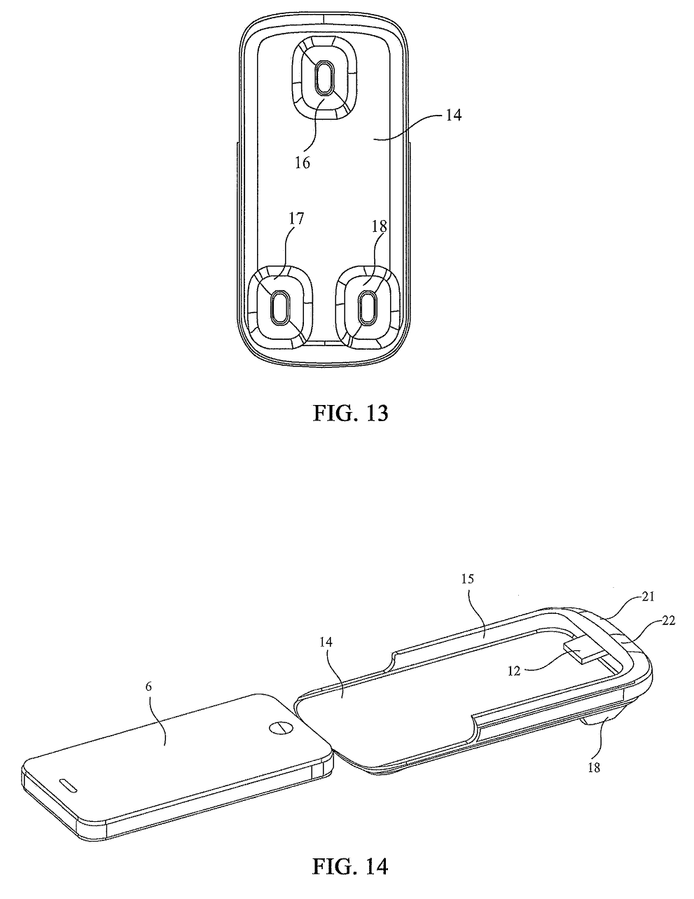

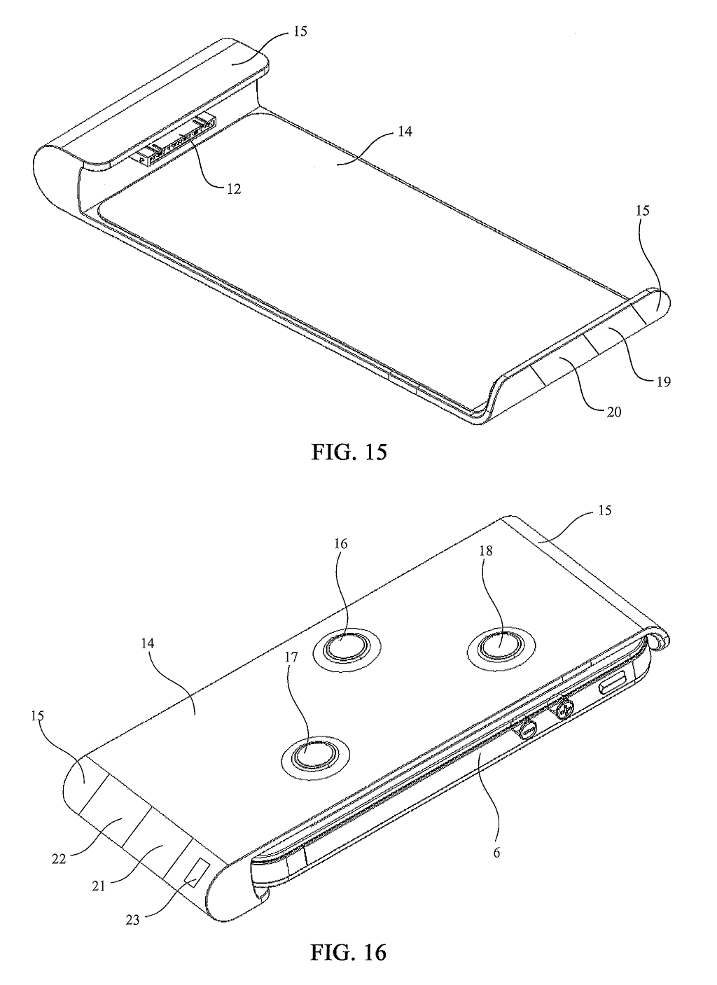

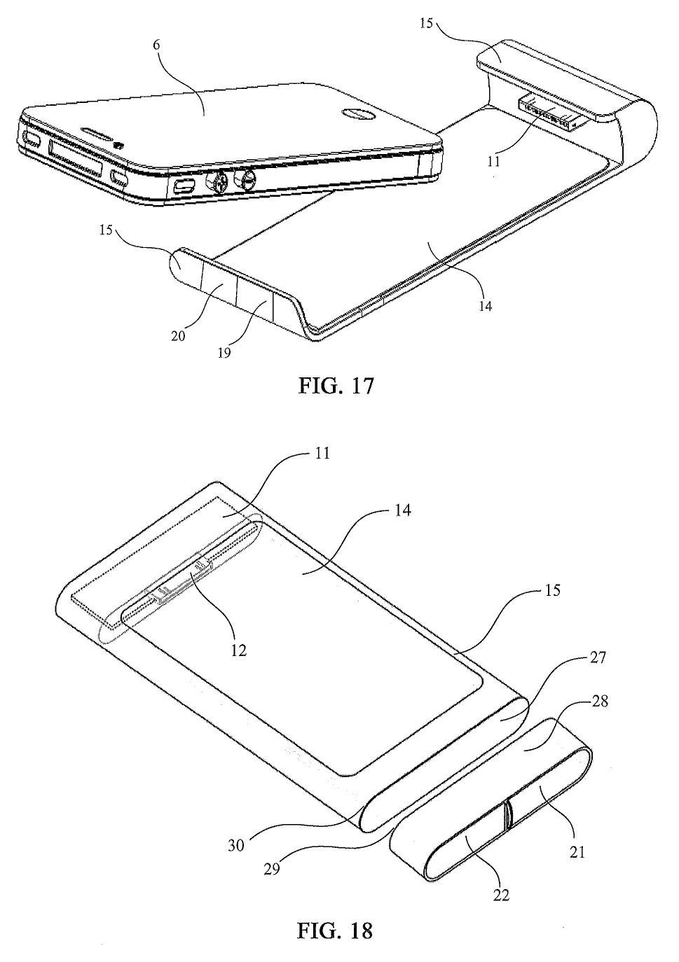

FIG. 6 is the front view of the structure of a measurement apparatus according to the second embodiment of the present invention, FIG. 7 is the back-side view of the structure of the measurement apparatus in FIG. 6, and FIG. 8 is a schematic view showing an application of the measurement apparatus in FIG. 6. As shown in FIGS. 6-8, the present embodiment differs from the above first embodiment in that: the measurement apparatus of the present embodiment further includes a first cover member 24 with an annular shape. The first cover member 24 is clasped on the sides 15, such that the terminal device 6 can be fitted in the cavity of the measurement apparatus more firmly. The other description is the same as in the first embodiment and is omitted here. The terminal communication interface 13 is not shown in the figures, and the descriptions thereof can be referred to FIG. 4.

In practice, the terminal device 6 is disposed in the cavity of the measurement apparatus, such that the measurement apparatus is sheathed outside the terminal device 6, and the terminal communication interface 13 is connected to the first data communication interface 12. For example, the terminal communication interface 13 can be plugged into the first data communication interface 12 directly, and the first cover member 24 is clasped on the sides 15. Then the user can measure the electrocardiograph by the measurement apparatus.

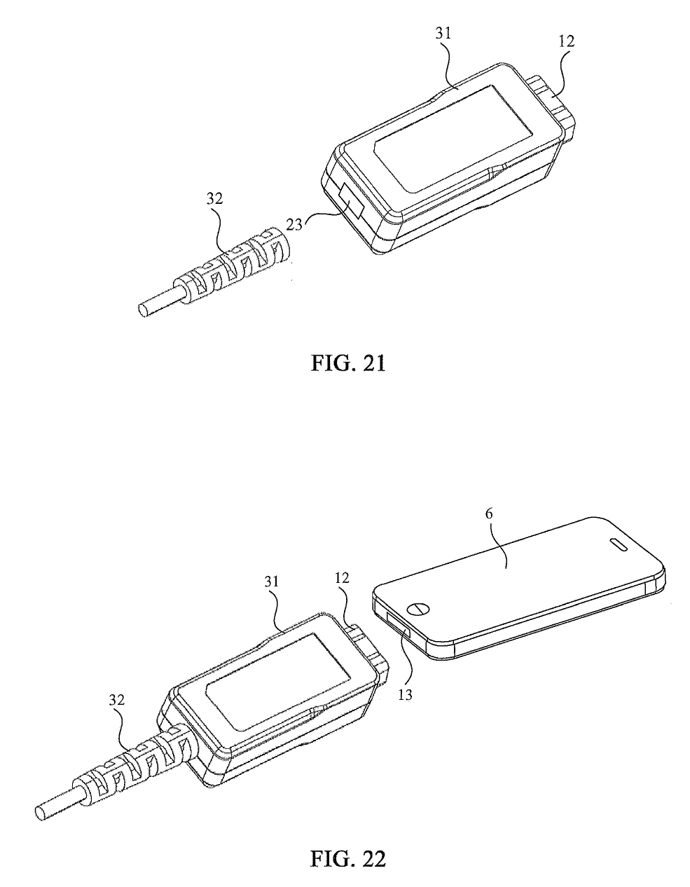

FIG. 9 is the front view of the structure of a measurement apparatus according to the third embodiment of the present invention, FIG. 10 is the back-side view of the structure of the measurement apparatus in FIG. 9, and FIG. 11 is a schematic view showing an application of the measurement apparatus in FIG. 9. As shown in FIGS. 9-11, the present embodiment differs from the above first embodiment in that: according to the present embodiment, chutes 25 are provided on two opposite edges of the bottom plate 14, and flanges 26 matching with the chutes 25 are provided on the sides 15 on the two opposite edges of the bottom plate 14, the sides 15 slide along the chutes 25 through the flanges 26, pulling a part of the sides 15 outwards to open the sides 15 and pushing the part of the sides 15 inwards to close the sides 15, such that the terminal device 6 can be placed in the cavity of the measurement apparatus. The other description is the same as in the first embodiment and is omitted here.

In practice, a part of the sides 15 is pulled outwards to open the sides 15, and the terminal device 6 is disposed in the cavity of the measurement apparatus, such that the measurement apparatus is sheathed outside the terminal device 6, and the terminal communication interface 13 is connected to the first data communication interface 12. For example, the terminal communication interface 13 can be plugged into the first data communication interface 12 directly, and the part of the sides 15 is pushed inwards to close the sides 15. Then the user can measure the electrocardiograph by the measurement apparatus.

FIG. 12 is the front view of the structure of a measurement apparatus according to the fourth embodiment of the present invention, FIG. 13 is the back-side view of the structure of the measurement apparatus in FIG. 12, and FIG. 14 is a schematic view showing an application of the measurement apparatus in FIG. 12. As shown in FIGS. 12-14, the present embodiment differs from the above first embodiment in that: the sides 15 are provided on one edge as well as the part of adjacent edges connected to the one edge of the bottom plate 14; and there are two contact electrodes disposed at the outside of the sides 15, i.e. the contact electrodes 21 and 22. The other description is the same as in the first embodiment and is omitted here. The terminal communication interface 13 is not shown in the figures, which is described with reference to FIG. 4.

In practice, the terminal device 6 is disposed in the cavity of the measurement apparatus, such that the measurement apparatus is sheathed outside the terminal device 6, and the terminal communication interface 13 is connected to the first data communication interface 12. For example, the terminal communication interface 13 can be plugged into the first data communication interface 12 directly. Then the user can measure the electrocardiograph by the measurement apparatus.

FIG. 15 is the front view of the structure of a measurement apparatus according to the fifth embodiment of the present invention, FIG. 16 is the back-side view of the structure of the measurement apparatus in FIG. 15, and FIG. 17 is a schematic view showing an application of the measurement apparatus in FIG. 15. As shown in FIGS. 15-17, the present embodiment differs from the above first embodiment in that: the sides 15 are provided on the two opposite edges of the bottom plate 14. The sides 15 have a upward bending structure. The other description is the same as in the first embodiment and is omitted here. The terminal communication interface 13 is not shown in the figures, which is described with reference to FIG. 4.

In practice, the terminal device 6 is disposed in the cavity of the measurement apparatus, such that the measurement apparatus is sheathed outside the terminal device 6, and the terminal communication interface 13 is connected to the first data communication interface 12. For example, the terminal communication interface 13 can be plugged into the first data communication interface 12 directly. Then the user can measure the electrocardiograph by the measurement apparatus.

FIG. 18 is the front view of the structure of a measurement apparatus according to the sixth embodiment of the present invention, FIG. 19 is the back-side view of the structure of the measurement apparatus in FIG. 18, and FIG. 20 is a schematic view showing an application of the measurement apparatus in FIG. 18. As shown in FIGS. 18-20, the present embodiment differs from the above first embodiment in that: in the present embodiment, one side on the bottom plate 14 is provide with an open 27; the main body further includes a second cover member 28, and the second cover member 28 is clasped on the open 27; the contact electrodes are disposed on the second cover member 28, a first conductive member 29 is disposed at the edge contacting the open 27 on the second cover member 28, and a second conductive member 30 is disposed on the open 27, the second conductive member 30 is connected to the signal processing module 11 through wires, the first conductive member 29 is in contact with the second conductive member 30 so as to achieve the electrical connection between the contact electrode on the second cover member 28 and the signal processing module 11. Preferably, the first conductive member 29 and the second conductive member 30 are made of metal (the wires are not shown). The contact electrodes on the second cover member 28 include the contact electrodes 21 and 22. The terminal communication interface 13 is not shown in the figures, and the descriptions thereof can be referred to FIG. 4. And the second data communication interface 23 is not shown in the figures, and the descriptions thereof can be referred to those in the first embodiment.

In practice, the terminal device 6 is disposed in the cavity of the measurement apparatus through the open 27, such that the measurement apparatus is sheathed outside the terminal device 6, and the terminal communication interface 13 is connected to the first data communication interface 12. For example, the terminal communication interface 13 can be plugged into the first data communication interface 12 directly. The second cover member 28 is clasped on the open 27, such that the first conductive member 29 on the second cover member 28 can contact the second conductive member 30 on the open 27. Then the user can measure the electrocardiograph by the measurement apparatus.

Preferably, in the embodiments of the present invention, there is a distance between the top surface of the contact electrodes disposed on the bottom plate 14 and the bottom plate 14, which means that the contact electrodes are higher than the bottom plate 14 for a certain height. Making the contact electrodes higher than the plane of the bottom plate 14 can ensure that each contact electrode can contact the human body stably, since the bottom plate 14 is flat and the outer appearance of human body is of an irregular curved surface. Specifically, there are three electrode mounting members for mounting the contact electrodes disposed on a plane outside of the bottom plate 14, and the three electrode mounting members are arranged in an isosceles triangle shape and higher than the plane outside of the bottom plate 14 for a distance. In particular, two electrode mounting members (the mounting members for mounting the contact electrodes 17 and 18) are disposed near the two ends of one side of the bottom plate 14, and the other electrode mounting member (the mounting member for mounting the contact electrode 16) is disposed at the center position on the other side of the bottom plate 14. Since the bottom plate 14 is flat while the outer appearance of human body is of a irregular curved surface, the electrode mounting members are set higher than the plane of the bottom plate 14 to make the contact electrode higher than the plane of the bottom plate 14, such that each of the contact electrodes can contact the human body stably. Screwing, riveting, clamping etc. can be used as the connection manners for connecting between the contact electrodes 16, 17, 18 and the respective electrode mounting members. In the present embodiment, screwing is employed as an example, i.e. one end of the contact electrode is processed to have thread, the end with thread enters from outside of the bottom plate 14, connects the corresponding lead wire with the contact electrode, and is fixed by a suitable nut. Thus, the height of the respective contact electrodes can be adjusted by changing the torsion depth of the nut, so as to adapt to the body characteristic of different users. Preferably, in the present embodiment, the end surfaces of the respective contact electrodes contacting human body are circular planes. However, the present invention is not limited thereto, for example the end surface of the contact electrode can be set to be square or of other polygon, or the central region of the end surface can be set to be a shape convex outwards or concave inwards, as long as the shape is adaptable to collect the physiological parameter signals on the skin.

In the embodiments of the present invention mentioned above, if the terminal device is a device with small volume such as a mobile phone, MP4 or MP3, the terminal device can be placed into the cavity of the measurement apparatus according to the solutions in the embodiments. If the terminal device is a device with a large volume such as a computer, then the terminal communication interface 13 can be connected with the first data communication interface 12 via a data wire, without placing the terminal device into the measurement apparatus. Therefore, in the present invention, the volume of the measurement apparatus can be designed to be portable, and when the volume of the terminal device is too large to be placed into the cavity of the measurement apparatus, the terminal device can be connected with the measurement apparatus through the data wire, so that the user can perform the electrocardiograph measurement using the measurement apparatus.

Furthermore, in the present embodiment, the first data communication interface 12 is a wireless interface and can be placed inside the main body, such that the first data communication interface 12 can be communicably connected with a wireless interface in the terminal device 6. For example, each of the wireless interfaces in the first data communication interface 12 and in the terminal device 6 can be bluetooth interface or infrared interface. In this situation, it is not necessary to place the terminal device 6 inside the measurement apparatus.

Furthermore, in the present embodiment, the second data communication interface 23 is a wireless interface and can be placed inside the main body, such that the second data communication interface 23 can be communicably connected with a wireless interface in the external detection device. For example, each of the wireless interfaces in the second data communication interface 23 and in the external detection device can be bluetooth interface or infrared interface. In this situation, it is not necessary to plug the external detection device into the second data communication interface 23.

In the present invention, the above-mentioned bottom plate 14 and the sides 15 can be made of soft glue material such as silica gel having the insulating property so as to ensure the contact electrodes insulating from each other. Alternatively, the material of the bottom plate 14 and the sides 15 can be hard plastic or textile, etc. The bottom plate 14 and the sides 15 of the measurement apparatus can be molded in one, or they can be manufactured separately and then combined together.

It should be noted that, the types of the external detection device are not limited to those described in the embodiments mentioned above, and can be added as required in actual applications.

It should be noted that, the number of the contact electrodes is not limited to the numbers described in the embodiments mentioned above, and can be altered as required in actual applications.

With the measurement apparatus of the present invention, various electrocardiograph measuring such as the hands measuring or the hands and ankles measuring as described in the background can be implemented without the hand-held electrocardiograph instrument. A chest measuring can also be implemented using the measurement apparatus of the present invention, specifically by placing the contact electrodes 16-18 of the measurement apparatus on the skin surface of human chest adjacent to the heart. For example, the three contact electrodes can be arranged around the heart, and preferably the three contact electrodes arranged in an isosceles triangle shape in the measurement apparatus can be placed at the left and right sides of the heart, and can be placed below the heart, specifically, the connection line between the contact electrodes 17 and 18 at the base angles of the isosceles triangle passes over the heart, and the other contact electrode 16 is placed right below the heart.

The measurement apparatus according to the above mentioned embodiments of the present invention includes a main body, a signal processing module disposed inside the main body, a first data communication interface coupled with the signal processing module, and a signal collection module coupled with the signal processing module, wherein the signal collection module outputs the measured physiological parameter signals to the signal processing module, the signal processing module processes the physiological parameter signals to generate the physiological parameter data and outputs the physiological parameter data to the terminal device via the first data communication interface, such that the terminal device can display the physiological parameter data. The user can conduct the physiological parameter measurement using the measurement apparatus according to the present invention without any specialized measurement equipment. The physiological parameter measurement can be implemented at any time and at any place only using the measurement apparatus according to the present invention together with various terminal devices carried at any time. Compared with the specialized electrocardiograph measurement instrument in the prior art, the measurement apparatus of the present invention has a reduced price, and since the measurement apparatus has a reduced volume, it is advantageous that the measurement apparatus is portable. There are contact electrodes disposed at the outside of the bottom plate of the measurement apparatus according to the embodiments of the present invention, such that the user can take a chest measuring manner when conducting the electrocardiograph measurement using the measurement apparatus of the present invention, i.e. by making the contact electrodes at the bottom plate sufficiently contact with the skin on heart of the patient. Such a manner can prevent the shortcomings in the prior art that the result of the electrocardiograph measurement is incorrect because of an improper posture in which the electrocardiograph measurement instrument is held by the user. Since the human chest is closest to the heart, the electrocardiograph signal is strongest, therefore a more accurate measurement result can be achieved with the chest measurement; besides, the chest measurement has no strict requirement on the posture that the user holds the measurement apparatus, so the usage of the hand-held electrocardiograph measurement instrument is simplified with ease. Therefore the present invention is advantageous by the simplicity and convenient operation. There is also a second data communication interface set on the main body of the measurement apparatus according to the embodiments of the present invention, and the external detection device can be connected with the second data communication interface, such that various physiological parameters can be measured and the extendibility of the measurement apparatus can be enhanced.

FIG. 21 is the schematic view of the structure of a measurement apparatus according to the seventh embodiment of the present invention, FIG. 22 is the schematic view of the application of the measurement apparatus in FIG. 21. As shown in FIGS. 21 and 22, the measurement apparatus includes: a main body, a signal processing module disposed inside the main body, a first data communication interface 12 coupled with the signal processing module, and a signal collection module coupled with the signal processing module. The first data communication interface 12 is coupled with a terminal device 6; the signal collection module functions to measure the physiological parameter signal and output the physiological parameter signal to the signal processing module; the signal processing module functions to process the physiological parameter signal to generate the physiological parameter data, and output the physiological parameter data to the terminal device via the first data communication interface 12, such that the terminal device 6 can display the physiological parameter data.

The detail of the terminal device 6 and the first data communication interface 12 in the present embodiment can be referred to that in the first embodiment.

In the present embodiment, the main body is a shell 31, the signal processing module is placed inside the shell 31. The shell 31 can be of an internal hollow structure. Preferably, the shell 31 is shaped to be a cube. Further, the shell 31 can adopt other shapes, which is not illustrated here. Specifically, the signal processing module is not shown in the figure since it is placed inside the shell 31; the detail is described with reference to the figure of the first embodiment.

In the present invention, the signal processing module can be directly powered by the terminal device 6 which is connected to the measurement apparatus. In particular, when the first data communication interface 12 is connected with the terminal communication interface 13, the terminal device 6 can power the signal processing module through the terminal communication interface 13 and the first data communication interface 12. Alternatively, the measurement apparatus can further include a power supply module provided inside the shell 31 and connected with the signal processing module, and the signal processing module is powered by the power supply module. For example, the power supply module (not shown) can be connected to the signal processing module through conducting wires. Preferably, the power supply module can be a button cell.

In the present embodiment, the signal collection module includes an external detection device 32, and the measurement apparatus further includes: a second data communication interface 23 which is disposed on the shell 31 and is connected to the signal processing module, the second data communication interface 23 functions to connect the external detection device 32. The external detection device 32 functions to measure the physiological parameter signal and output the physiological parameter signal to the signal processing module through the second data communication interface 23. The external detection device 32 can be plugged into the second data communication interface 23 directly, at this time the communication interface of the external detection device 32 needs to match the second data communication interface 23. The second data communication interface 23 can be a standard interface, e.g. a USB interface, or a specialized interface for a certain external detection device 32. The external detection device 32 can be separated from the shell 31, and when used, the external detection device 32 can be plugged into the second data communication interface 23 of the shell 31, as shown in FIG. 21.

Optionally, the external detection device 32 can be a blood oxygen measurement module, a fetal heart measurement module, a temperature measurement module or an inductive electrode. Details of the blood oxygen measurement module, the fetal heart measurement module, the temperature measurement module and the inductive electrode as well as the corresponding signal processing module can be referred to the first embodiment, and the specific description thereof is omitted here.

Furthermore, in the present embodiment, the first data communication interface 12 is a wireless interface and can be placed inside the shell 31, such that the first data communication interface 12 can be communicably connected with a wireless interface in the terminal device 6. For example, each of the wireless interfaces in the first data communication interface 12 and in the terminal device 6 can be bluetooth interface or infrared interface. In this situation, it is not necessary to plug the terminal device 6 into the first data communication interface 12.

Furthermore, in the present embodiment, the second data communication interface 23 is a wireless interface and can be placed inside the shell 31, such that the second data communication interface 23 can be communicably connected with a wireless interface in the external detection device 32. For example, each of the wireless interfaces in the second data communication interface 23 and in the external detection device can be bluetooth interface or infrared interface. In this situation, it is not necessary to plug the external detection device 32 into the second data communication interface 23.

In the present embodiment, the above-mentioned shell 31 can be made of soft glue material such as the silica gel. Alternatively, the material of the shell 31 can be hard plastic or textile.

It should be noted that, the types of the external detection device are not limited to those described in the embodiments mentioned above, and can be added as required in actual applications.

The measurement apparatus according to the above mentioned embodiments of the present invention includes: a main body, a signal processing module disposed inside the main body, a first data communication interface coupled with the signal processing module, and a signal collection module coupled with the signal processing module. The signal collection module functions to measure the physiological parameter signal and output the physiological parameter signal to the signal processing module; the signal processing module functions to process the physiological parameter signal to generate the physiological parameter data, and output the physiological parameter data to the terminal device via the first data communication interface, such that the terminal device can display the physiological parameter data. The user can directly conduct the physiological parameter measurement using the measurement apparatus according to the present invention without any specialized measurement equipment. The measurement apparatus according to the present invention together with various hand-held terminal devices carried at any time can implement the physiological parameter measurement at any time and at any place. Compared with the specialized electrocardiograph measurement instrument in the prior art, the measurement apparatus of the present invention has a reduced price, and since the measurement apparatus has a reduced volume, it is advantageous that the measurement apparatus is portable. There is also a second data communication interface set on the main body of the measurement apparatus according to the above embodiments of the present invention, and the external detection device can be connected to the second data communication interface, such that various physiological parameters can be measured and the extendibility of the measurement apparatus can be enhanced.

It should be appreciated that the above embodiments are only exemplary embodiments for explaining the principle of the invention, not for limiting the invention thereto. The persons skilled in the art can make various modifications and variations without departing from the scope and the spirit of the invention, and the modifications and variations also drop within the protective scope of the present invention.

1. A measurement apparatus, comprising: a main body, a signal processing module disposed inside the main body, a first data communication interface coupled with the signal processing module, and a signal collection module coupled with the signal processing module; wherein
the first data communication interface is coupled with a terminal device;
the signal collection module functions to measure the physiological parameter signal and output the physiological parameter signal to the signal processing module; and
the signal processing module functions to process the physiological parameter signal to generate the physiological parameter data, and output the physiological parameter data to the terminal device via the first data communication interface, such that the terminal device can display the physiological parameter data.

2. The measurement apparatus of claim 1, wherein the main body includes a bottom plate and sides disposed at edges of the bottom plate.

3. The measurement apparatus of claim 2, wherein the sides have an internal hollow structure, and the signal processing module is disposed inside the sides.

4. The measurement apparatus of claim 2, wherein a projection portion is provided at the outside of the bottom plate, and the signal processing module is disposed inside the projection portion.

5. The measurement apparatus of claim 2, wherein the sides and the bottom plate form a cavity, and the main body is nested outside of the terminal device through the cavity.

6. The measurement apparatus of claim 5, wherein the sides surround at the edges of the bottom plate.

7. The measurement apparatus of claim 6, wherein the measurement apparatus further includes a first cover member with an annular shape, and the first cover member is clasped on the sides.

8. The measurement apparatus of claim 6, wherein chutes are provided in two opposite edges of the bottom plate, and flanges matching with the chutes are provided at the two opposite edges of the bottom plate, the sides slide along the chutes through the flanges.

9. The measurement apparatus of claim 5, wherein the sides are provided at one edge and a part of an edge adjacent to the one edge of the bottom plate.

10. The measurement apparatus of claim 5, wherein the sides are provided at the two opposite edges of the bottom plate.

11. The measurement apparatus of claim 6, wherein one side of the bottom plate has an open.

12. The measurement apparatus of claim 11, wherein the main body has a second cover member, and the second cover member is clasped on the open.

13. The measurement apparatus of claim 12, wherein the signal collection module includes at least two contact electrodes, and the contact electrodes are disposed on the second cover member; and
a first conductive member is disposed at the edge contacting the open on the second cover member, and a second conductive member is disposed on the open, the second conductive member is connected to the signal processing module through wires, the first conductive member is in contact with the second conductive member so as to achieve the electrical connection between the contact electrode on the second cover member and the signal processing module.

14. The measurement apparatus of claim 1, wherein the main body is a shell and the signal processing module is disposed inside the shell.

15. The measurement apparatus of claim 1, wherein the measurement apparatus further includes a power supply module which is disposed inside the main body and connected to the signal processing module; and
the power supply module functions to supply power to the signal processing module.

16. The measurement apparatus of claim 1, wherein the first data communication interface is disposed inside the main body, and the first data communication interface is a wireless interface.

17. The measurement apparatus of claim 2, wherein the signal collection module includes at least two contact electrodes, and the physiological parameter signal includes the electrocardiograph signal, the physiological parameter data includes the electrocardiograph data, and the signal processing module includes an electrocardiograph processing sub-module;
the contact electrodes function to measure the electrocardiograph signal, and output the electrocardiograph signal to the electrocardiograph processing sub-module; and
the electrocardiograph processing sub-module functions to process the electrocardiograph signal to generate the electrocardiograph data, and output the electrocardiograph data to the terminal device through the first data communication interface, such that the terminal device can display the electrocardiograph data.

18. The measurement apparatus of claim 17, wherein three contact electrodes are disposed at the outside of the bottom plate, and the three contact electrodes are arranged in an isosceles triangle shape.

19. The measurement apparatus of claim 17, wherein the contact electrodes are disposed at the outside of the sides.

20. The measurement apparatus of claim 17, wherein the contact electrodes are connected to the signal processing module through wires, and the sides have an internal hollow structure, and the wires are disposed inside the sides.

21. The measurement apparatus of claim 1, wherein the signal collection module includes an external detection device, and the measurement apparatus further includes: a second data communication interface which is disposed on the main body and is connected to the signal processing module, the second data communication interface functions to connect the external detection device;
the external detection device functions to measure the physiological parameter signal and output the physiological parameter signal to the signal processing module through the second data communication interface.

22. The measurement apparatus of claim 21, wherein the external detection device is a blood oxygen measurement module, the physiological parameter signal includes the blood oxygen signal, the physiological parameter data includes the blood oxygen data, and the signal processing module includes a blood oxygen processing sub-module;
the blood oxygen measurement module functions to measure the blood oxygen signal and output the blood oxygen signal to the blood oxygen processing sub-module; and
the blood oxygen processing sub-module functions to process the blood oxygen signal to generate the blood oxygen data, and output the blood oxygen data to the terminal device through the first data communication interface, such that the terminal device can display the blood oxygen data.

23. The measurement apparatus of claim 21, wherein the external detection device is a fetal heart measurement module, and the physiological parameter signal includes the fetal heart signal, the physiological parameter data includes the fetal heart data, and the signal processing module includes a fetal heart processing sub-module;
the fetal heart measurement module functions to measure the fetal heart signal, and output the fetal heart signal to the fetal heart processing sub-module; and
the fetal heart processing sub-module functions to process the fetal heart signal to generate the fetal heart data, and output the fetal heart data to the terminal device through the first data communication interface, such that the terminal device can display the fetal heart data.

24. The measurement apparatus of claim 21, wherein the external detection device is a temperature measurement module, the physiological parameter signal includes the temperature signal, the physiological parameter data includes the temperature data, and the signal processing module includes a temperature processing sub-module;
the temperature measurement module functions to measure the temperature signal and output the temperature signal to the temperature processing sub-module; and
the temperature processing sub-module functions to process the temperature signal to generate the temperature data, and output the temperature data to the terminal device through the first data communication interface, such that the terminal device can display the temperature data.

25. The measurement apparatus of claim 21, wherein the external detection device is an inductive electrode, the physiological parameter signal includes the electrocardiograph signal, the physiological parameter data includes the electrocardiograph data, and the signal processing module includes an electrocardiograph processing sub-module;
the inductive electrode functions to measure the electrocardiograph signal, and output the electrocardiograph signal to the electrocardiograph processing sub-module; and
the electrocardiograph processing sub-module functions to process the electrocardiograph signal to generate the electrocardiograph data, and output the electrocardiograph data to the terminal device through the first data communication interface, such that the terminal device can display the electrocardiograph data.

26. The measurement apparatus of claim 21, wherein the second data communication interface is disposed inside the main body, and the second data communication interface is a wireless interface.
